(19) United States
(12) Patent Application Publication
(10) Pub. No.: US 2023/0323130 A1
(43) Pub. Date: Oct. 12, 2023

(54) METHODS OF PRODUCING DYES WITH VARIOUS HUE FROM HUITO FRUIT

(71) Applicant: Archer Daniels Midland Company, Decatur, IL (US)

(72) Inventors: Jielu Zhao, Cincinnati, OH (US); Gregory Horn, Cincinnati, OH (US)

(73) Assignee: Archer Daniels Midland Company, Decatur, IL (US)

(21) Appl. No.: 18/042,981

(22) PCT Filed: Aug. 26, 2021
(86) PCT No.: PCT/US2021/047762

§ 371 Date: Feb. 24, 2023

Related U.S. Application Data
(60) Provisional application No. 63/070,489, filed on Aug. 26, 2020.

(51) Int. Cl. C09B 61/00; C09B 67/20

(52) U.S. Cl. CPC C09B 61/00; C09B 67/0065

(57) ABSTRACT

A method of forming a colorant having a desired hue comprises mixing a component of a Huito fruit with an amino acid, thus forming a reaction mixture wherein the component of Huito fruit reacts with the amino acid and produces a blue color, and adjusting the hue of the blue color by adjusting the amount of oxygen present during reaction of the component of Huito fruit and the amino acid. The method may comprise adjusting a temperature of the mixing and/or other processing parameters.

### FIELD OF THE INVENTION

This invention relates to methods of producing dyes with various hue from Huito fruit.

### BACKGROUND OF THE INVENTION

Today, synthetic chemicals, such as colorants or cross-linking reagents, tend to have decreasing acceptance in the food, cosmetic, animal feed and textile industries. For safety reasons, whether real or perceived, people tend to favor the use of natural or organic ingredients in food, cosmetic, textile, and biomaterial products.

Genipin is a colorless compound. It belongs to the iridoid group. It is very active chemically and reacts immediately when combined with compounds having primary amine groups, such as amino acids, collagen, chitosan, glucosamine-type compounds and various proteins and enzymes. When oxygen is present, the product may turn to blue, green, or black quickly. Genipin is an iridoid ester, therefore, it can be hydrolyzed to generate genipinic acid which also can react with different compounds to generate red and brown colorants. The colorants generated from genipin are heat and pH stable. Since genipin normally comes from plant materials, its Kosher characteristics provide great potential for use of genipin-derived colorants in bakery and canned food applications.

Genipin and other iridoid compounds, such as genipinic acid, genipin-gentiobioside, geniposide and geniposidic acid, are found in the fruits and leaves of Genipa americana, also known as Genipap, or Huito, a tropical wild plant. Genipin is naturally present in the mature fruit, and its quantity is from 0 to 3.0% of fruit weight depending on the degree of ripeness. Genipin is stable in the plant cell even though it is not established where it is stored. Whenever the cell is broken, genipin will react spontaneously with the amino acids that naturally exist in the fruit pulp and turn color to blue or black in an air environment.

U.S. Pat. No. 8,557,319 discloses a method of preparing colored products comprising processing Genipa americana fruit juice, which contains genipin, genipin derivatives, or pre-genipin compounds, with other edible juices or extracts which contain nitrogenous compounds such as amino acids, polypeptides, or proteins.

U.S. Pat. No. 8,945,640 discloses a method of manufacturing a blue colorant by using the genipin-rich extract reaction and mixing with water and amino acids (for example, lysine, histidine, arginine, glutamine, asparagine, methionine, glycine, glutamic acids, tyrosine, valine, alanine, serine, leucine, taurine, carnitine, ornithine and citrulline, in the presence of oxygen. The patent discloses that the blue shades generated are variable among deep blue, violet-blue, bright-blue, and greenish-blue depending on the amino acid used.

U.S. Pat. No. 7,927,637 discloses a method to make a blue colorant, wherein the blue colorant is derived from unprocessed raw juice obtained from Genipa americana fruit pulp, and wherein said raw juice is mixed with glycine (liquid) or with glycine plus starch (powder). The reference discloses that except for an additional step of warming up the juice-glycine mix, and in the case of the powder further dehydration of the juice-glycine-starch remix, no further steps are required to make a temperature and pH stable blue colorant.

CN 105624198 discloses a method for preparing gardenia blue pigment in different hues. The reference discloses that the method includes the following steps: hydrolysis reaction, polymerization reaction, separation and purification, dry molding, and verification. In the hydrolysis reaction, the raw material gardenoside is hydrolyzed with beta-glucoside at a pH of about 8-8.3 (with pH adjustment obtained by adding sodium hydroxide (NaOH), wherein the solution is heated to 50° C. with a 50° C. water bath). In the polymerization reaction, the hydrolyzed gardenoside is polymerized with an amino acid wherein an oxidant is introduced into the reaction vessel, and the temperature of the water bath is increased to 70° C. The reference discloses that the oxidizing agent includes compressed air, pure oxygen, hydrogen peroxide (H₂O₂) and other oxidizing agents which can be used in foods.

Food manufacturers increasingly desire natural alternatives to synthetic colorants. It would be beneficial to have processes that can produce food colorants derived from natural ingredients, wherein the food colorants have a wide variety of blue color hues and color intensity strength. Conventional methods are limited in that they do not provide the ability to fine tune production of such food colorants. It would be beneficial to have processes and products that do not have the disadvantages of conventional methods and products.

### SUMMARY

The present invention provides improvements over conventional methods and products. In an aspect, a process for forming a colorant having a desired hue comprises mixing a component of Huito fruit with an amino acid, thus forming a reaction mixture wherein the component of Huito fruit reacts with the amino acid and produces a blue color, and adjusting the hue of the blue color by adjusting the amount of oxygen present during the reaction of the component of Huito fruit and the amino acid. As used herein, the term “adjusting the oxygen present” means having a predetermined amount of oxygen present during reaction of the component of Huito fruit and the amino acid. In an embodiment, the adjusting the oxygen present comprises having a predetermined amount of air present during the reaction component of Huito fruit and the amino acid the component of Huito fruit and the amino acid.

In an aspect, the adjusting the hue of the color further comprises heating the reaction mixture of the component of Huito fruit and the amino acid at a predetermined reaction temperature for a predetermined period of time. In an embodiment, the predetermined reaction temperature is 45° C. to 95° C. and the predetermined period of time is 1 to 24 hours. In a more preferred embodiment, the predetermined reaction temperature is 50° C. to 95° C. and the predetermined period of time is 2 to 20 hours. In an even more preferred embodiment, the predetermined reaction temperature is 60° C. to 90° C., for example about 80° C., and the predetermined period of time is 4 to 14 hours.

In an aspect, the amino acid is chosen from the group consisting of taurine, glutamic acid, glycine, isoleucine, asparagine, serine, aspartic acid, phenylalanine, alanine, and glutamine. In an aspect, the adjusting the hue of the blue color further comprises selecting an amino acid from this group.

In an aspect, the adjusting the hue of the blue color comprises mixing a predetermined ratio of the component of Huito fruit and amino acid.

In an aspect, a method comprises adjusting both the hue and strength of the blue color by adjusting the amount of oxygen present during the reaction of a component of Huito fruit and an amino acid.

These and other aspects, embodiments, and associated advantages will become apparent from the following Detailed Description.

### DETAILED DESCRIPTION

The present invention relates to methods of controlling the hue of dyes generated from mixing Huito fruit and various amino acids. In an aspect, the present disclosure shows that by using different amino acids, hues ranging from violet to turquoise can be obtained. In an aspect, methods are provided wherein oxygen levels are adjusted, resulting into a bathochromic shift in the resulting color. In an aspect, the timing and duration of air introduction and the rate of oxygen flow can be manipulated to achieve dye with desired hue.

In an aspect, temperature during the reaction of Huito fruit component and an amino acid is adjusted, thereby providing an adjustable parameter to vary the level of dissolved oxygen in aqueous solution, which in turn, allows for production of a dye with a desired amount of color and hue. Temperature relates inversely to level of dissolved oxygen in aqueous solution. Thus, higher temperature leads to formation of dyes with bluer hue. In an aspect, it is shown that a lower amount of solvent leads to bathochromic shift.

Through manipulation of the above reaction parameters, dye products with desired hues and color intensity strength can be achieved with high yield and purity. This approach facilitates the production of products with balanced performance and production cost. Moreover, dyes with different hues ranging from violet to turquoise can be produced to meet different commercial needs.

Aspects of the present invention include forming a colorant having a desired hue in methods wherein Huito fruit is mixed with an amino acid with oxygen present to produce a blue color, and adjusting the hue of the blue color, wherein the adjusting comprises adjusting the oxygen present. In an embodiment, the adjusting the hue of the blue color comprises adjusting the oxygen present by adjusting the amount of air present, wherein the air is bubbled through a reaction mixture of Huito fruit and the amino acid.

In an aspect, the method comprises mixing Huito fruit with a particular amino acid with oxygen present.

In an aspect, the adjusting of the hue of the blue color comprises adjusting the oxygen present by adjusting the amount of oxygen or air being bubbled through the reaction mixture of Huito fruit and an amino acid.

In an aspect, the adjusting of the hue of the blue color comprises adjusting the oxygen present by adjusting the amount of air present, wherein exposure to air is solely by surface area exposure of the reaction mixture of Huito fruit and an amino acid to air.

In an aspect, the adjusting of the hue of the blue color further comprises adjusting the temperature of the mixture of Huito fruit and an amino acid.

In an aspect, the adjusting of the hue of the blue color further comprises mixing a solvent with the Huito fruit and an amino acid, and adjusting the amount of solvent present in the mixture. In an aspect, the solvent is deionized water (DI). In an aspect, the component of Huito fruit is Huito juice obtained by cutting Huito fruit in half, and pressing a cut half of Huito fruit with a fruit press. The ratio by weight of amino acid to Huito fruit in the reaction mixture may be adjusted to obtain a desired color. It has been found that as the ratio of amino acid to Huito fruit is increased in the reaction mixture, a higher color value is obtained, but at some level, the increase in the amount of amino acid to Huito fruit results in a diminished or no further return on the increase in color value and may not be justified in view of cost of amino acid. In an aspect, the ratio by weight of Huito fruit to the amino acid in the reaction mixture is in the range of 10:1 to 400:1, more preferably in the range of 80:1 to 120:1, more preferably in the range of 90:1 to 110:1, e.g., about 100:1. In an aspect, the component of Huito fruit is obtained by cutting Huito fruit into more than two pieces. The pieces of cut Huito fruit may be blended with deionized water to form a fruit-water blend. The ratio by weight of cut Huito fruit to deionized water in the fruit-water blend may be in the range of 1:0.1 to 1:100, more preferably in the range of 1:0.5 to 1:50, more preferably in the range of 1:1 to 1:10, more preferably 1:3 to 1:5, e.g., about 1:4 The pH of the reaction mixture may be adjusted, e.g., to pH 5 to 8, more preferably 6 to 7.8, and even more preferably 6.5 to 7.5, such as about 7, and this pH adjustment may be made with a base, e.g., aqueous NaOH.

The above aspects and other aspects of the present invention are described further in the examples below.

### Example 1

In this example, different amino acids were tested for forming a colorant having a desired hue comprises mixing a component of a Huito fruit with an amino acid with oxygen present.

Step 1—550 g frozen Huito fruit was thawed, peeled and cut into small pieces and blended with 2200 g deionized (hereinafter, DI) water with a Ninja food blender. Deionized water was used to avoid impact of ions.

Step 2—200 g of this puree was put into each of eleven individual beakers. To flask #1 was added 0.4 g taurine, flask #2 0.4 g L-glutamic acid, flask #3 0.4 g glycine, and flask #4 0.4 g L-isoleucine, flask #5 0.4 g L-asparagine, flask #6 0.4 g L-serine, flask #7 0.4 g Aspartic acid, flask #8 0.4 g L-phenylalanine, flask #9 0.4 g alanine, flask #10 0.4 g glutamine. The mixtures were adjusted to pH=7 with aqueous NaOH.

Step 3—The flasks were then placed in a water bath pre-heated to 40° C. and incubated for 1 hour. The puree in each flask was filtered through #3 filter paper. The greenish-blue cloudy solutions were adjusted to pH=7 and placed into a water bath preheated to 70° C. Compressed air supplied with an aquarium air pump was bubbled through the bottom of the solutions for 6 hours.

Step 4—The reaction solutions were brought to 100 g total weight with DI water.

Step 5—The color value (CU^{1%}), i.e., color intensity, and hue (λ,ₘₐₓ) of the dye solutions were measured with Perkin Elmer Lambda 20 UV-Vis Spectrophotometer, and the results as shown in Table 1.

TABLE 1

| Amino Acids | λ,ₘₐₓ (nm), Hue | CU^{1%} |
|---|---|---|
| Taurine | 588, violet-blue | 1.45 |
| L-glutamic acid | 593, blue | 0.61 |
| Glycine | 581, violet | 2.40 |
| L-isoleucine | 596, blue | 1.02 |
| L-asparagine | 588, violet-blue | 1.35 |
| L-serine | 588, violet-blue | 1.67 |
| L-Aspartic acid | 590, blue | 0.62 |
| L-phenylalanine | 595, blue | 1.19 |
| L-alanine | 584, violet | 1.59 |
| L-glutamine | 592, blue | 1.12 |

### Example 2

Methods of Incorporating Oxygen (Bubbling Air Versus Bubbling Pure Oxygen).

Step 1—400 g frozen Huito fruit was thawed, peeled and cut into small pieces and blended with 1600 g DI water in a Ninja food blender. The resulting puree was incubated in 40° C. water bath for 1 hour and filtered through #3 filter paper with a Buchner funnel. Moderate pressure was applied to the residue to facilitate filtration near the end of the filtering process. The filtrate was collected as a cloudy greenish-blue liquid (1600 mL) and used as is in next step.

Step 2—200 g of the Huito solution from step 1 was placed into each of three Erlenmeyer flasks equipped with magnetic stir bars. L-alanine (0.5 g) was added to each flask. The solutions were adjusted to pH=7 with aqueous NaOH.

Step 3—The reaction flasks were placed onto a Thermo Scientific multi-position hotplate and heated to 70° C. while stirring.

Step 4—Compressed air was bubbled through the bottom of flask #1 into the solution with an aquarium air pump. Oxygen was bubbled through the bottom of flask #2 in the solution with an oxygen cylinder. Reaction solution of flask #3 was open to atmosphere.

Step 5—The reactions were allowed to continue for 6 hours and water was added to restore the original volumes, i.e., 200 g. Color hue (λₘₐₓ) and color values (CU^{1%}) of the resulting dye solutions were evaluated with Perkin Elmer Lambda 20 UV-Vis Spectrophotometer, and the results are shown in Table 2.

Step 6—All three reactions produced dye with different hues and color values. The solution in flask #3 exhibited a blue hue with (λₘₐₓ)=595 nm and color value (CU^{1%}) 0.64. The flask #2 solution resulted from oxygen bubbling was a violet hue with (λₘₐₓ)=578 nm and color value (CU^{1%}) 1.35, while the flask #1 solution resulted from bubbling air was a violet-blue hue with (λₘₐₓ)=584 nm and color value (CU^{1%}) 1.70. As shown in Table 2, by adjusting the amount of oxygen present during the reaction of the component of Huito fruit and the amino acid so as to increase the amount of oxygen present during the reaction, the color value, i.e., color intensity, was substantially increased. As shown in Table 2, bubbling pure oxygen through the bottom of flask #2 resulted in the flask #2 solution having color value (CU^{1%}) 1.35, and bubbling air through the bottom of flask #1 resulted in the flask #1 solution having color value (CU^{1%}) 1.70, whereas, with merely exposing the flask #3 solution to the atmosphere (and no bubbling of pure oxygen or air) resulted in the flask #3 solution having color value (CU^{1%}) 0.64.

TABLE 2

| Methods of O₂ | | |
|---|---|---|
| Incorporation | λ, max (nm), Hue | CU^{1%} |
| Bubbling air | 584, violet | 1.70 |
| Bubbling oxygen | 578, violet | 1.35 |
| No bubbling of air/oxygen | 595, blue | 0.64 |

### Example 3

Methods and Duration of Incorporating Oxygen (Surface Exposure Vs Bubbling into Solution).

Step 1—100 g Huito water extract prepared as described in step 1 of example 2 was placed into each of four 250 mL beakers.

Step 2—To beakers #1 and #2 was added 0.2 g L-alanine each; to beakers #3 and #4 was added 0.328 g L-glutamine each.

Step 3—The solutions in the four beakers were adjusted to pH=7 with aqueous NaOH and heated to 80° C.

Step 4—With a 4-port aquarium air pump, air was bubbled into the solutions in beakers #1 and #3, respectively, through the bottom of the beakers and onto the surfaces of the solutions in beakers #2 and #4 respectively.

Step 5—The reaction solutions were maintained at pH=7 and heated at 80° C. for 8 hours.

Step 6—The reaction solutions were brought to the original volumes, i.e., 100 g total weight with DI water.

Step 7—The solutions were measured with Perkin Elmer Lambda 20 UV-Vis Spectrophotometer for hue and color values, and the results are shown in Table 3. As shown in Table 3, alanine as the amino acid resulted in greater color value, i.e., intensity, than glutamine as the amino acid. Table 3 shows how the hue and color value may be fine-tuned to obtain a desired hue and color value by selecting a particular amino acid, using air bubbling or no air bubbling, and selecting a particular reaction duration.

TABLE 3

| | Color (CU^{1%}, λₘₐₓ) vs. Reaction Duration | | | | |
|---|---|---|---|---|---|
| Reaction | 4 h | 8 h | 12 h | 15 h | Hue |
| #1 (alanine, | 0.83, | 1.15, | 1.35, | 1.32, | Violet |
| bubbling) | 591 nm | 588 nm | 588 nm | 584 nm | |
| #2 (alanine, no | 0.55, | 0.85, | 1.22, | 1.21, | Violet- |
| bubbling) | 592 nm | 592 nm | 588 nm | 588 nm | blue |
| #3 (glutamine, | 0.67, | 0.85, | 1.14, | 1.15, | Blue |
| bubbling) | 595 nm | 592 nm | 590 nm | 590 nm | |
| #4 (glutamine, | 0.49, | 0.73, | 1.16, | 1.15, | Blue |
| no bubbling) | 597 nm | 595 nm | 591 nm | 591 nm | |

### Example 4

Impact of Temperature

Step 1—400 g frozen Huito fruit was thawed, peeled and cut into small pieces and blended with 1600 g DI water in a Ninja food blender. The resulting puree was incubated in 40° C. water bath for 1 hour and filtered through #3 filter paper with a Buchner funnel. Moderate pressure was applied to the residue to facilitate filtration near the end of the filtering process. The filtrate was collected as a cloudy greenish-blue liquid (1655 mL). The filtrate was further filtered through Celite coated filter paper to obtain a clear solution.

Step 2—A 3-neck round bottom flask equipped with magnetic stir bar was charged with 100 g Huito solution and 0.4 g L-glutamine. Aqueous NaOH was used to adjust pH=7.

Step 3—The reaction mixture was heated to 90° C. with a heating mantle. Air was bubbled into the solution with a fish tank air pump. The reaction was allowed to continue for 10 hours while maintaining pH=7.

Step 4—Heat source was removed, and the reaction was allowed to cool down, and deionized water was added to restore the original volumes, i.e., 100 g as described in Step 2. Color value (CU^{1%}) and hue (λₘₐₓ) of the resulting dye product was measured with Perkin Elmer Lambda 20 UV-Vis Spectrophotometer.

Step 5—Three more reactions were performed as described in steps 2-4 above with reaction temperatures of 80° C., 70° C. and 60° C., respectively, and the results shown in Table 4. Table 4 shows how the hue and color value may be fine-tuned to obtain a desired hue and color value by selecting a particular reaction temperature. The lowest reaction temperature of this example (i.e., 60° C.) resulted in the greatest color value as compared to higher reaction temperatures (i.e., 70° C., 80° C., and 90° C.).

TABLE 4

| Reaction Temperature | λₘₐₓ (nm), Hue | CU^{1%} |
|---|---|---|
| 60° C. | 583, violet | 1.90 |
| 70° C. | 587, violet-blue | 1.50 |
| 80° C. | 588, violet-blue | 1.42 |
| 90° C. | 588, violet-blue | 1.46 |

### Example 5

Amount of Solvent with L-Alanine

Step 1—I. Frozen Huito fruits 1000 g was thawed and peeled. All fruits were cut in halves and split into two 500 g batches. One batch (500 g) was juiced with a fruit press and 340 g of Huito juice was obtained.

Step 2—The other batch of fruit was cut into small pieces and split into three identical sub-batches.
a) Batch #1 167 g fruit was blended with 167 g DI water in a Ninja food blender for five minutes. The resulting puree was incubated in a water bath for one hour at 40° C. and filtered to obtain a greenish blue cloudy solution as Huito extract #1 (210 g).
b) Batch #2 167 g fruit was blended with 334 g DI water in a Ninja food blender for five minutes. The resulting puree was incubated in a water bath for one hour at 40° C. and filtered to obtain a greenish blue cloudy solution as Huito extract #2 (351 g).
c) Batch #3 167 g fruit was blended with 668 g DI water in a Ninja food blender for five minutes. The resulting puree was incubated in a water bath for one hour at 40° C. and filtered to obtain a greenish blue cloudy solution as Huito extract #3 (703 g).

Step 3—Each of the four fruit juice or extracts obtained in steps 1 and 2 was used to react with L-alanine in four separate Erlenmeyer flasks as follows, wherein the ratio of fruit juice or extract to amino acid L-alanine is maintained at 100:1.
a) Reaction #1: 100 g juice from step 1 mixed with 1 g L-alanine.
b) Reaction #2: 100 g extract from step 2a mixed with 0.5 g L-alanine.
c) Reaction #3: 100 g extract from step 2b mixed with 0.33 g L-alanine.

d) Reaction #4: 100 g extract from step 2c mixed with 0.2 g L-alanine.

Step 4. —The reaction solutions were adjusted to pH=7 with aqueous NaOH and heated to 80° C. with a Thermo Scientific multi-position hotplate stirrer. Air was bubbled into from the bottom of the solutions with an aquarium air pump. The reactions were allowed to continue for 14 hours, with deionized water added to restore the original volumes, i.e., 100 g, prior to each monitoring of color value (CU 1%) and hue (max), at 4 hours, 8 hours, 12 hours, and 14 hours, and the results shown in Table 5. Table 5 shows how the hue and color value may be fine-tuned to obtain a desired hue and color value by selecting a particular amount of solvent (here, deionized water) and a particular amount of amino acid (here, the exemplary amino acid being L-alanine).

TABLE 5

| | Color (CU^{1%}, λₘₐₓ) vs. Reaction Duration | | | | |
|---|---|---|---|---|---|
| Reactions | 4 h | 8 h | 12 h | 14 h | Hue |
| #1 | 3.64, | 5.79, | 6.84, | 7.13, | Violet-blue |
| | 593 nm | 591 nm | 591 nm | 588 nm | |
| #2 | 2.73, | 4.28, | 4.35, | 4.29, | Violet-blue |
| | 590 nm | 587 nm | 587 nm | 587 nm | |
| #3 | 2.05, | 2.53, | 2.33, | 2.26, | Violet-blue |
| | 590 nm | 588 nm | 587 nm | 587 nm | |
| #4 | 1.71, | 1.73, | 1.50, | 1.46, | Violet |
| | 583 nm | 584 nm | 583 nm | 584 nm | |

### Example 6

Amount of Solvent with L-Glutamine

Step 1—Frozen Huito fruit 500 g was thawed, peeled and cut into small pieces. This was further shredded into small particles with a Ninja food blender. The shredded Huito was then blended with DI water in the following portions.
#1-150 g fruit with 150 g water.
#2-100 g fruit with 200 g water.
#3-75 g fruit with 225 g water.
#4-60 g fruit with 240 g water.

Step 2—The purees obtained in step 1 was incubated in a water bath at 40° C. for one hour and filtered off. Greenish-blue solutions were obtained as follows.
#1-210 g
#2-240 g
#3-248 g
#4-259 g

Step 3—Reactions between Huito extracts obtained in step 2 and L-glutamine were set up in four separate Erlenmeyer flasks in the following ways.
a) Reaction #1: 100 g extract mixed with 1.071 g L-glutamine.
b) Reaction #2: 100 g extract mixed with 0.625 g L-glutamine.
c) Reaction #3: 100 g extract mixed with 0.453 g L-glutamine.
d) Reaction #4: 100 g extract mixed with 0.347 g L-glutamine.

Step 4. —The reaction solutions were adjusted to pH=7 with aqueous NaOH and heated to 80° C. with a Thermo Scientific multi-position hotplate stirrer. Air was bubbled into from the bottom of the solutions with a fish tank air pump. The reactions were allowed to continue for 8 hours and the resulting dye solutions were brought back to the original volumes, i.e., 100 g with DI water. The product dye solutions were measured with Perkin Elmer Lambda 20 UV-Vis Spectrophotometer and Menolta CR-400 Chroma Meter, and the data (CU^{1%}, λ,ₘₐₓ, L, a, b) are shown in Table 6. Table 6 shows how the hue and color value may be fine-tuned to obtain a desired hue and color value by selecting a ratio of Huito fruit to solvent (here, deionized water), and a particular amount of amino acid (here, the exemplary amino acid being L-glutamine).

TABLE 6

| Reactions | λₘₐₓ | CU^{1%} | L | a | b |
|---|---|---|---|---|---|
| #1 | 588 nm | 0.98 | 43.16 | 1.22 | −10.06 |
| #2 | 588 nm | 0.50 | 42.93 | 1.58 | −9.59 |
| #3 | 588 nm | 0.38 | 42.76 | 1.65 | −9.34 |
| #4 | 588 nm | 0.25 | 42.65 | 1.71 | −9.14 |

### Example 7

Amount of Amino Acid

Step 1—Frozen Huito fruit 400 g was thawed, peeled and cut into small pieces. The Huito fruit was blended with 1600 g DI water with a Ninja food blender for 5 minutes. The greenish paste obtained was filtered through coarse filter paper and 1600 g extract was obtained.

Step 2—200 g Huito extract obtained in step 1 was added to four (4) Erlenmeyer flasks, with specific amounts of L-alanine as described as follows.
Flask #1-0.215 g L-alanine
Flask #2-0.307 g L-alanine
Flask #3-0.461 g L-alanine
Flask #4-0.614 g L-alanine

Step 3—Aqueous NaOH was used to adjust the solutions in each flask to pH=7. The four flasks were put on a Thermo Scientific multi-position hotplate stirrer and heated to 80° C. while stirring. Air was bubbled through the bottom of the flasks with a multi-channel aquarium pump. Temperature was maintained at 80° C. with a temperature probe and the pH's in each flask was adjusted to 7 after every 30 minutes. The reactions were allowed to continue for 8 hours and the resulting dye solutions were brought back to the original volumes, i.e., 200 g with DI water. The product dye solutions were measured with Perkin Elmer Lambda 20 UV-Vis Spectrophotometer. Table 7 shows how the hue and color value varied based on the amount of L-alanine used. As the ratio of amino acid to Huito fruit is increased in the reaction mixture, a higher color value is obtained. When the ratio of amino acid to Huito fruit is increased in the reaction mixture, comparing Reaction #1 (with 0.215 g L-alanine) and Reaction #4 (with 0.614 L-alanine), the wavelength decreased.

TABLE 7

| | Color (CU^{1%}, λₘₐₓ) vs. Reaction Duration | | | | |
|---|---|---|---|---|---|
| Reactions | 2 h | 4 h | 6 h | 8 h | Hue |
| #1 | 0.53, | 0.74, | 0.90, | 0.98, | Violet-blue |
| | 588 nm | 588 nm | 587 nm | 588 nm | |
| #2 | 0.78, | 1.17, | 1.44, | 1.48, | Violet |
| | 587 nm | 585 nm | 584 nm | 584 nm | |
| #3 | 0.81, | 1.22, | 1.77, | 1.60, | Violet |
| | 590 nm | 587 nm | 586 nm | 585 nm | |
| #4 | 0.99, | 1.38, | 1.86, | 1.87, | Violet |
| | 585 nm | 584 nm | 583 nm | 583 nm | |

Those having skill in the art, with the knowledge gained from the present disclosure, will recognize that various changes can be made to the disclosed processes in attaining these and other advantages, without departing from the scope of the present disclosure. As such, it should be understood that the features of the disclosure are susceptible to modifications and/or substitutions. The specific embodiments illustrated and described herein are for illustrative purposes only, and not limiting of the invention as set forth in the appended claims.

1. A method comprising:
a) mixing a component of Huito fruit with an amino acid, thus forming a reaction mixture wherein the component of Huito fruit reacts with the amino acid and produces a blue color; and
b) adjusting the hue of the blue color by adjusting the amount of oxygen present during reaction of the component of Huito fruit and the amino acid, thereby forming a colorant having a desired hue.

2. The method of claim 1, wherein the adjusting the amount of oxygen present during the reaction comprises adjusting the amount of air present during the reaction of the component of Huito fruit and the amino acid.

3. The method of claim 1, wherein the adjusting the amount of oxygen present during the reaction consists of one of exposing a surface area of the reaction mixture to pure oxygen, exposing a surface area of the reaction mixture to pure oxygen and bubbling pure oxygen into the reaction mixture, exposing a surface area of the reaction mixture to air, or exposing a surface area of the reaction mixture to air and bubbling air into the reaction mixture.

4. The method of claim 1, wherein the adjusting the hue of the blue color further comprises heating the reaction mixture at a predetermined reaction temperature for a predetermined period of time.

5. The method of claim 4, wherein the predetermined reaction temperature is 45° C. to 95° C. and the predetermined period of time is 1 to 24 hours.

6-7. (canceled)

8. The method of claim 1, wherein the amino acid is chosen from taurine, glutamic acid, glycine, isoleucine, asparagine, serine, aspartic acid, phenylalanine, alanine, and glutamine.

9. The method of claim 1, wherein the component of Huito fruit is Huito juice.

10. The method of claim 1, wherein the component of Huito fruit is Huito juice obtained by cutting Huito fruit in half, and pressing a cut half of Huito fruit with a fruit press.

11. The method of claim 1, wherein the ratio by weight of Huito fruit to the amino acid in the reaction mixture is in the range of 10:1 to 400:1.

12-14. (canceled)

15. The method of claim 1, wherein the component of Huito fruit is obtained by cutting Huito fruit into more than two pieces.

16. The method of claim 15, wherein the pieces of cut Huito fruit are blended with deionized water to form a fruit-water blend.

17. The method of claim 16, wherein the ratio by weight of cut Huito fruit to deionized water in the fruit-water blend is in the range of 1:0.1 to 1:100.

18-21. (canceled)

22. The method of claim 17, wherein the reaction mixture is adjusted to pH 5 to 8.

23. The method of claim 22, wherein the reaction mixture is adjusted to pH 5 to 8 with aqueous NaOH.

24. The method of claim 1, wherein the hue (λₘₐₓ) of the blue color is adjusted to one of 588 nm violet-blue, 593 nm blue, 581 nm violet, 596 nm blue, 590 nm blue, 595 nm blue, 584 nm violet, 592 nm blue, 578 nm violet, 583 nm violet, and 587 nm violet-blue.

25. The method of claim 1, wherein the hue (λₘₐₓ) of the blue color is adjusted in the range of 575 nm violet to 615 nm blue.

26. The method of claim 1, wherein the hue (λₘₐₓ) of the blue color is adjusted in the range of 575 nm violet to 605 nm blue.

27. The method of claim 1, wherein the hue (λₘₐₓ) of the blue color is adjusted in the range of 578 nm violet to 595 nm blue.

28. A method comprising:
mixing a component of Huito fruit with an amino acid, thus forming a reaction mixture wherein the component of Huito fruit reacts with the amino acid and produces a blue color; and
adjusting the hue and color value of the blue color by adjusting the amount of oxygen present during reaction of the component of Huito fruit and the amino acid, thereby forming a colorant having a desired hue and color value.

29. The method of claim 28, wherein the formed colorant has a hue (max) in the range of 575 nm violet to 615 nm blue, wherein the colorant has a greater color value with increasing the amount of oxygen present during the reaction than the color value without an increase in the amount of oxygen present during the reaction.
